(19) United States
(12) Patent Application Publication
(10) Pub. No.: US 2023/0329664 A1
(43) Pub. Date: Oct. 19, 2023

(54) SLIP RING ASSEMBLY, MEDICAL SYSTEM, AND METHOD THEREOF

(71) Applicant: SHANGHAI UNITED IMAGING HEALTHCARE CO., LTD., Shanghai (CN)

(72) Inventors: Hongtao WANG, Shanghai (CN); Tao LIU, Shanghai (CN); Cheng NI, Shanghai (CN); Li WANG, Shanghai (CN); Hongjun ZHANG, Shanghai (CN); Wei ZHANG, Shanghai (CN); Tuota XING, Shanghai (CN); Buliang SUN, Shanghai (CN); Yansi YANG, Shanghai (CN); Hualing WANG, Shanghai (CN); Shaomin XIAO, Shanghai (CN)

(73) Assignee: SHANGHAI UNITED IMAGING HEALTHCARE CO., LTD., Shanghai (CN)

(21) Appl. No.: 18/332,767

(22) Filed: Jun. 12, 2023

Related U.S. Application Data
(63) Continuation of application No. PCT/CN2021/138130, filed on Dec. 14, 2021.

(30) Foreign Application Priority Data
Dec. 14, 2020 (CN) 202011468108.5
Sep. 1, 2021 (CN) 202111021712.8

(51) Int. Cl. A61B 6/00; H01R 39/10

(52) U.S. Cl. CPC A61B 6/56; H01R 39/10; H01R 2201/12

(57) ABSTRACT

The present disclosure relates to a slip ring assembly. The slip ring assembly may include a first slip ring, a second slip ring, and a transmission component. The transmission component may be configured to facilitate at least one of: data transmission of the first slip ring, data transmission of the second slip ring, power transmission of the first slip ring, or power transmission of the second slip ring.

### CROSS-REFERENCE TO THE RELATED APPLICATIONS

This application is a Continuation of International Application No. PCT/CN2021/138130, filed on Dec. 14, 2021, which claims priority of Chinese Application No. 202011468108.5 filed on Dec. 14, 2020, and Chinese Application No. 202111021712.8 filed on Sep. 1, 2021, the contents of each of which are hereby incorporated by reference.

### TECHNICAL FIELD

The present disclosure generally relates to medical technology, and more particularly, slip ring assemblies and/or medical systems.

### BACKGROUND

An existing medical system may be a multi-modality one including multiple medical devices (e.g., a treatment device, an imaging device, multiple imaging devices of different modalities). Various components of such a medical system may be connected through cables, and the wiring of the cables may be complex. For example, an image-guided radiation therapy (IGRT) system includes a treatment device and an imaging device. The imaging device may be used to view a specific target tissue (e.g., cancerous tissue) before, during, or after a treatment device delivers radiation treatment to the target tissue. In an existing IGRT system, the imaging device is connected to the treatment device through cables. Thus, it is desirable to develop a system for simply connecting multiple medical devices of a medical system.

### SUMMARY

According to one aspect of the present disclosure, a slip ring assembly may be provided. The slip ring assembly may include a first slip ring, a second slip ring, and a transmission component. The transmission component may be configured to facilitate at least one of: data transmission of the first slip ring, data transmission of the second slip ring, power transmission of the first slip ring, or power transmission of the second slip ring.

In some embodiments, the transmission component may be configured to transmit at least one signal of the first slip ring and at least one signal of the second slip ring.

In some embodiments, the transmission component may include a static ring.

In some embodiments, at least two of the first slip ring, the second slip ring, or the static ring may wind around each other.

In some embodiments, at least two of the first slip ring, the second slip ring, or the static ring may be located in a same plane.

In some embodiments, the static ring may be located between the first slip ring and the second slip ring.

In some embodiments, a radius of the static ring may be larger than a radius of the first slip ring. A radius of the second slip ring may be larger than the radius of the static ring.

In some embodiments, the transmission component may include a first carbon brush assembly and a second carbon brush assembly. The first carbon brush assembly may be operably connected to the first slip ring and configured to facilitate at least one of first data transmission or first power transmission between the first slip ring and the transmission component. The second carbon brush assembly may be operably connected to the second slip ring and configured to facilitate at least one of second data transmission or second power transmission between the second slip ring and the transmission component.

In some embodiments, the slip ring assembly may include a carbon brush assembly. The carbon brush assembly may be configured to facilitate: at least one of first data transmission or first power transmission between the first slip ring and the transmission component, and at least one of second data transmission or second power transmission between the second slip ring and the transmission component.

In some embodiments, the transmission component may include at least one transmission module and at least one receiving module that are configured to facilitate: first contactless data transmission between the first slip ring and the transmission component, or second contactless data transmission between the second slip ring and the transmission component.

In some embodiments, the at least one transmission module may include a first transmission module and a second transmission module. The at least one receiving module may include a first receiving module and a second receiving module. The first transmission module and the first receiving module may be configured to facilitate the first contactless data transmission between the first slip ring and the transmission component. The second transmission module and the second receiving module may be configured to facilitate the second contactless data transmission between the second slip ring and the transmission component.

In some embodiments, the at least one transmission module may also include a third transmission module and a fourth transmission module that are different from the first transmission module and the second transmission module, respectively. The at least one receiving module may also include a third receiving module and a fourth receiving module that are different from the first receiving module and the second receiving module, respectively. The third transmission module and the third receiving module may also be configured to facilitate the first contactless data transmission between the first slip ring and the transmission component. The fourth transmission module and the fourth receiving module may also be configured to facilitate the second contactless data transmission between the second slip ring and the transmission component.

In some embodiments, one transmission module of the at least one transmission module may be opposingly positioned with respect to one receiving module of the at least one receiving module that corresponds to the transmission module.

In some embodiments, a distance between the transmission module and the corresponding receiving module may be smaller than a distance threshold.

In some embodiments, one of the at least one transmission module may include a transmitter and an antenna.

In some embodiments, at least one of the first contactless data transmission or the second contactless data transmission may be implemented according to a communication protocol.

In some embodiments, the communication protocol may include peripheral component interconnect express (PCIe).

In some embodiments, a speed of at least one of the first contactless data transmission or the second contactless data transmission may exceed a speed threshold.

In some embodiments, the slip ring assembly may also include a second transmission component. The transmission component may be configured to transmit at least one signal of the first slip ring. The second transmission component may be configured to transmit at least one signal of the second slip ring.

In some embodiments, the transmission component may include at least one carbon brush assembly. The at least one carbon brush assembly may be operably connected to the first slip ring and the second slip ring. The at least one carbon brush assembly may be configured to facilitate: at least one of first data transmission or first power transmission between the first slip ring and the transmission component, and at least one of second data transmission or second power transmission between the second slip ring and the transmission component.

In some embodiments, the transmission component may include a first static ring. The second transmission component may include a second static ring that is different from the first static ring. At least two of the first slip ring, the second slip ring, the first static ring, or the second static ring may be disposed coaxially.

In some embodiments, the transmission component may include at least one transmission module and at least one receiving module that are configured to facilitate first contactless data transmission between the first slip ring and the transmission component.

In some embodiments, the at least one transmission module may include a first transmission module and a second transmission module. The at least one receiving module may include a first receiving module and a second receiving module. The first transmission module and the first receiving module may be configured to facilitate the first contactless data transmission between the first slip ring and the transmission component. The second transmission module and the second receiving module may also be configured to facilitate the first contactless data transmission between the first slip ring and the transmission component.

In some embodiments, the second transmission component may include at least one transmission module and at least one receiving module that are configured to facilitate second contactless data transmission between the second slip ring and the transmission component.

In some embodiments, the at least one transmission module may include a first transmission module and a second transmission module. The at least one receiving module may include a first receiving module and a second receiving module. The first transmission module and the first receiving module may be configured to facilitate the second contactless data transmission between the second slip ring and the second transmission component. The second transmission module and the second receiving module may also be configured to facilitate the second contactless data transmission between the second slip ring and the second transmission component.

According to another aspect of the present disclosure, a system may be provided. The system may include a first gantry, a second gantry, a fixed gantry, and a slip ring assembly. The first gantry may be configured to accommodate at least a portion of a first imaging assembly. The second gantry may be configured to accommodate at least a portion of a second imaging assembly or at least a portion of a treatment assembly. The fixed gantry may be configured to support the first gantry or the second gantry. The slip ring assembly may be configured to facilitate data transmission of the first imaging assembly, the second imaging assembly, or the treatment assembly. The slip ring assembly may include a first slip ring located on the first gantry. The slip ring assembly may include a second slip ring located on the second gantry. The slip ring assembly may include a transmission component located on the fixed gantry. The transmission component may be configured to facilliate at least one of: data transmission of the at least a portion of the first imaging assembly, data transmission of the at least a portion of the second imaging assembly or the at least a portion of the treatment assembly, power transmission of the at least a portion of the first imaging assembly, or power transmission of the at least a portion of the second imaging assembly or the at least a portion of the treatment assembly.

In some embodiments, the data transmission may include contact data transmission or contactless data transmission.

In some embodiments, the data transmission may include bidirectional data transmission.

In some embodiments, the transmission component may be configured to transmit: at least one signal of the at least a portion of the first imaging assembly, and at least one signal of the at least a portion of the second imaging assembly or the at least a portion of the treatment assembly.

In some embodiments, at least two of the first slip ring, the second slip ring, or the transmission component may wind around each other.

In some embodiments, at least two of the first slip ring, the second slip ring, or the transmission component may be located in a same plane.

In some embodiments, a static ring of the transmission component may be located between the first slip ring and the second slip ring.

In some embodiments, the system may also include a second transmission component. The transmission component may be configured to transmit at least one signal of the at least a portion of the first imaging assembly. The second transmission component may be configured to transmit at least one signal of the at least a portion of the second imaging assembly or the at least a portion of the treatment assembly.

In some embodiments, at least two of the first slip ring, the second slip ring, the transmission component, or the second transmission component may be disposed coaxially.

In some embodiments, the first slip ring and the transmission component may be located on one side of the first gantry or the second gantry, and the second slip ring and the second transmission component may be located on another side of the first gantry or the second gantry along an axial direction of the first gantry or an axial direction of the second gantry.

In some embodiments, the first slip ring, the transmission component, the second slip ring, and the second transmission component may be located on a same side of the first gantry or the second gantry along an axial direction of the first gantry or an axial direction of the second gantry.

In some embodiments, at least a portion of the first gantry may be housed in the second gantry. The first gantry may be rotatably connected to the second gantry. The second gantry may be rotatably connected to the fixed gantry.

In some embodiments, the first gantry may be rotatable along a first axis. The second gantry may be rotatable along a second axis. The first axis may intersect the second axis.

In some embodiments, the first gantry may be rotatable along a first axis. The second gantry may be rotatable along a second axis. The first axis may be parallel to the second axis.

In some embodiments, the system may also include a lock mechanism configured to lock the first gantry and the second gantry. The lock mechanism may be located on the first gantry, on the second gantry, or between the first gantry and the second gantry.

According to another aspect of the present disclosure, a method for operating a system may be provided. The system may include a first medical assembly, a second medical assembly, and a slip ring assembly. The slip ring assembly may be configured to facilitate data transmission of the first medical assembly, data transmission of the second medical assembly, power transmission of the first slip ring assembly, or power transmission of the second slip ring assembly. The method may include: obtaining data of a first portion of the first medical assembly or a first portion of the second medical assembly; and transmitting, through the slip ring assembly, the data to a second portion of the first medical assembly, a second portion of the second medical assembly, or a control component of the system.

In some embodiments, the data may include imaging data of a subject or treatment data of the subject acquired by the first medical assembly or the second medical assembly.

In some embodiments, the transmitting, through the slip ring assembly, the data to a second portion of the first medical assembly, a second portion of the second medical assembly, or a control component of the system may include: transmitting the data through a contact data transmission mode.

In some embodiments, the transmitting, through the slip ring assembly, the data to a second portion of the first medical assembly, a second portion of the second medical assembly, or a control component of the system may include: transmitting the data through a contactless data transmission mode.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 and FIG. 3 are schematic diagrams illustrating an exemplary medical device according to some embodiments of the present disclosure;

FIG. 4 is a section view illustrating an exemplary medical device according to some embodiments of the present disclosure;

FIG. 5 is a schematic diagram illustrating an exemplary slip ring assembly according to some embodiments of the present disclosure;

FIG. 6A and FIG. 6B illustrate two views of an exemplary slip ring assembly according to some embodiments of the present disclosure;

FIG. 6C illustrates a section view of the exemplary slip ring assembly along AA axis in FIG. 6A according to some embodiments of the present disclosure; and

FIG. 7 is a section view illustrating an exemplary medical device according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that the terms “system,” “device,” “assembly,” “component,” etc., when used in this disclosure, refer to one or more parts with one or more specific purposes. However, a structure that may perform a same or similar function compared to a part exemplified above or referred to elsewhere in the present disclosure may be named differently from the present disclosure.

In the present disclosure, spatial reference terms such as “center,” “longitudinal,” “transverse,” “length,” “width,” “thickness,” “upper,” “lower,” “front,” “back,” “left,” “right,” “vertical,” “horizontal,” “top,” “bottom,” “inner,” “outer,” “clockwise,” “counterclockwise,” “axial,” “radial,” “circumferential,” etc., indicate, in a relative sense, an orientation or positional relationship between two or more elements, assemblies, devices, or systems based on an orientation or positional relationship as shown in the drawings, and are only for the convenience and simplicity of description, rather than indicating or implying that the elements, assemblies, devices or systems in the present disclosure have a particular orientation when the disclosed system, or a portion thereof, is in operation, or are constructed and operated in a particular orientation, and therefore may be not understood as a limitation of the present disclosure.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention.

In the present disclosure, unless otherwise clearly specified and limited, the terms “mount,” “connect,” “couple,” “fix,” “locate,” “dispose,” etc., should be understood in a broad sense, for example, it may be a fixed connection, a detachable connection, integrated into a whole, a mechanical connection, an electrical connection, directly connected, or indirectly connected via an intermediate medium, an internal connection of two elements, or an interconnection of two elements, unless otherwise clearly defined. For those skilled in the art, the specific meanings of the above terms in the present disclosure may be understood according to specific circumstances.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

An aspect of the present disclosure relates to a slip ring assembly. The slip ring assembly may include a first slip ring, a second slip ring, and a transmission component located between the first slip ring and the second slip ring. The transmission component may be operably connected to the first slip ring and the second slip ring and configured to transmit data of the first slip ring and data of the second slip ring.

In some embodiments, the slip ring assembly may be configured in a medical system. The slip ring assembly may be configured to supply power to and/or facilitate data transmission of at least one component (e.g., an imaging assembly, a treatment assembly) of the medical system, thereby obliviating the need to employ cables to implement power supply to and/or data transmission between different medical assemblies (e.g., an imaging assembly and a treatment assembly, imaging assemblies of different modalities) as in an existing medical system.

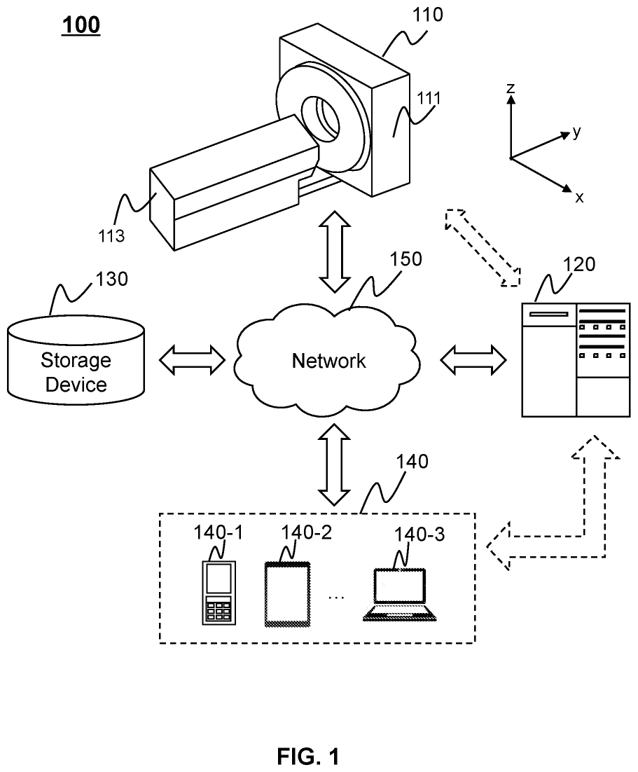

In addition, by obliviating the need to employ cables and complex wiring thereof for connecting devices or assemblies of a medical system for power supply and/or data transmission, the slip ring assembly may reduce the dimension along an axial direction (e.g., the y-direction as illustrated in FIG. 1) of the medical system, or a portion thereof.

In addition, by allowing the first slip ring and the second slip ring to share the transmission component, the slip ring assembly may also reduce the dimension along a radial direction (e.g., the z-direction as illustrated in FIG. 1) of the medical system, or a portion thereof, thereby causing the imaging source and/or treatment source closer to a subject (e.g., a patient) in the medical system. By reducing the distance of the pathway that the imaging medium and/or treatment medium (e.g., X-ray) traverses before impinging on the subject, or a portion thereof, the spreading of the imaging medium and/or treatment medium along the pathway may be reduced, the accuracy of the delivery of the imaging medium and/or the treatment medium may be improved, and/or the image accuracy and/or treatment efficacy based on the imaging medium or treatment medium may be improved.

In some embodiments, the transmission component may include a static ring. The static ring may be positioned between the first slip ring and the second slip ring, thereby enhancing the reliability of data transmission between the first (or second) slip ring and the static ring. Besides, the slip ring assembly may be configured with both a contact data transmission mode and a contactless data transmission mode. The accuracy of the data transmitted in one of the two data transmission modes may be validated by the data transmitted in the other data transmission mode. When one of the two data transmission modes fails to function properly, data transmission may proceed in the other data transmission mode, thereby further improving the reliability of data transmission via the slip ring assembly.

FIG. 1 is a schematic diagram illustrating an exemplary medical system according to some embodiments of the present disclosure.

As illustrated in FIG. 1, the medical system 100 may include a medical device 110, a processing device 120, a storage device 130, one or more terminals 140, and a network 150. The components in the medical system 100 may be connected in one or more of various ways. Merely by way of example, the medical device 110 may be connected to the processing device 120 through the network 150. As another example, the medical device 110 may be connected to the processing device 120 directly as indicated by the bi-directional arrow in dotted lines linking the medical device 110 and the processing device 120. As a further example, the storage device 130 may be connected to the processing device 120 directly or through the network 150. As still a further example, the terminal 140 may be connected to the processing device 120 directly (as indicated by the bi-directional arrow in dotted lines linking the terminal 140 and the processing device 120) or through the network 150.

In some embodiments, the medical system 100 may include a first medical assembly and a second medical assembly. In some embodiments, each of the first medical assembly and the second medical assembly may include an imaging assembly (also referred to as an imaging device), respectively. The imaging assembly may be configured to perform an imaging on a target volume (also referred to as a target region, a region, e.g., a tumor, a lesion) of a subject (e.g., a patient).

In some embodiments, the first medical assembly may include a first imaging assembly, and the second medical assembly may include a second imaging assembly or the treatment assembly. The first imaging assembly may be the same as or different from the second imaging assembly. In some embodiments, each of the first medical assembly and the second medical assembly may include a treatment assembly (also referred to as a treatment device). The treatment assembly may be configured to deliver a treatment beam to the target volume to perform a radiotherapy (e.g., stereotactic radiosurgery and/or precision radiotherapy) on the target volume. In some embodiments, the first medical assembly may include the imaging assembly, and the second medical assembly may include the treatment assembly.

In some embodiments, the medical system 100 may perform image-guided radiation therapy (IGRT) that monitors and/or treat the target volume. In this case, the medical device 110 may include the treatment assembly and the imaging assembly. The imaging assembly may perform the imaging of the target volume and/or normal tissue surrounding the target volume before, after, or while the radiotherapy is performed. In this way, the anatomy, as well as the motion or deformation, of the target volume can be detected, and the patient's position and/or the treatment beam can be adjusted for more precise radiation dose delivery to the target volume.

In some embodiments, the imaging assembly may perform the imaging by emitting an imaging beam, e.g., an X-ray beam, a y ray beam, an ultrasonic wave, etc., towards the subject. For example, the imaging assembly may include a computed tomography (CT) imaging device (e.g., a cone-beam computed tomography (CBCT) device, a fan-beam computed tomography (FBCT) device), a magnetic resonance imaging (MRI) device, a positron emission tomography (PET) device, a single-photon emission computerized tomography (SPECT) device, a PET-CT imaging device, an X-ray machine, a digital radiology (DR) imaging device, an ultrasonic imaging device, or the like, or any combination thereof.

In some embodiments, the imaging assembly may include various components including, e.g., at least one imaging source, at least one imaging detector corresponding to the at least one imaging source, etc. The at least one imaging source may be configured to deliver at least one imaging beam towards the subject. The at least one imaging detector may be configured to detect at least a portion of the at least one imaging beam. In some embodiments, the imaging assembly may include a tube, a high-voltage device, etc. The tube and/or the high-voltage device may be configured to facilitate a delivery of the at least one imaging beam. For example, the tube may be configured to generate the at least one imaging beam. The high-voltage device may accelerate an electron beam to generate the at least one imaging beam.

In some embodiments, the treatment assembly may be an electromagnetic device that is configured to accelerate charged particles to a higher-energy state under an electric field. In some embodiments, the treatment assembly may include a linear accelerator. The linear accelerator may generate an X-ray beam, an electron beam, a proton beam, etc., to treat the target volume.

In some embodiments, the treatment assembly may include a treatment head, a treatment detector (e.g., an electronic portal imaging device (EP ID)), etc. The treatment head may be configured to deliver a treatment beam toward the subject to perform the radiation treatment and/or imaging of the target volume. The treatment detector may be configured to detect at least a portion of the treatment beam.

In some embodiments, the treatment head may include a treatment source (e.g., a target), a microwave device, an acceleration device (e.g., an acceleration tube), a cooling device, a collimator, a filter (e.g., a flattening filter), a chamber, etc. The treatment source may be configured to deliver the treatment beam towards the subject. The acceleration device may be configured to accelerate an electron beam to generate the treatment beam. The microwave device may be configured to facilitate the delivery of the treatment beam. For example, the microwave device may generate an electromagnetic field for accelerating the electron beam to a relatively high energy. The cooling device may be configured to cool at least one component of the treatment head (e.g., the microwave device, the acceleration device). The collimator may be configured to adjust a radiation range of the treatment beam. The filter may be configured to generate a filtered treatment beam by adjusting an energy distribution of the treatment beam. The chamber may be configured to ionize gas in the chamber to detect at least one parameter (e.g., an intensity, a flatness, a symmetry) of the treatment beam.

In the present disclosure, the x-axis, the y-axis, and the z-axis shown in FIG. 1 may form an orthogonal coordinate system. The x-axis and the y-axis shown in FIG. 1 may be horizontal, and the z-axis may be vertical. As illustrated, the positive x-direction along the x-axis may be from the left side to the right side of the medical device 110 seen from the direction facing the front of the medical device 110; the positive z-direction along the z-axis shown in FIG. 1 may be from the lower part to the upper part of the medical device 110; the positive y-direction along the y-axis shown in FIG. 1 may refer to a direction in which a subject is moved into a bore of the medical device 110.

In some embodiments, the medical device 110 may include a gantry assembly 111. The gantry assembly 111 may be configured to support at least one component of the imaging assembly and/or at least one component of the treatment assembly, for example, the treatment head, the at least one imaging source, the at least one imaging detector, the treatment detector, etc. At least a portion of the gantry assembly 111 (e.g., a first gantry, a second gantry) may be configured to rotate around the subject (e.g., a patient, or a portion thereof) that is moved into or located within a field of view (FOV) (e.g., a region covered by at least one radiation beam emitted from at least one of the treatment head or the at least one imaging source) of the medical device 110.

In some embodiments, the gantry assembly 111 may include a first gantry (e.g., an annular structure), a second gantry (e.g., an annular structure), and a fixed gantry (e.g., an annular structure). The first gantry may be configured to accommodate at least a first portion of the first medical assembly (e.g., the imaging assembly, the treatment assembly) or at least a first portion of the second medical assembly (e.g., the imaging assembly, the treatment assembly). The second gantry may be configured to accommodate at least a second portion of the first medical assembly or at least a second portion of the second medical assembly. The fixed gantry may be configured to support the first gantry and the second gantry. In some embodiments, the first medical assembly may be located on the first gantry, and the second medical assembly may be located on the second gantry. In some embodiments, a portion of the first medical assembly may be located on the first gantry and a portion on the second gantry. In some embodiments, a portion of the second medical assembly may be located on the first gantry and a portion on the second gantry. For example, the first medical device may include the imaging assembly, and the second medical assembly may include the treatment assembly. The at least one imaging source, the at least one imaging detector, a portion of the treatment head (e.g., the collimator), or the treatment detector may be located on the first gantry, and a portion of the treatment head (e.g., the treatment source, the microwave device, the acceleration device, the cooling device) may be located on the second gantry.

In some embodiments, at least a portion of the imaging assembly and at least a portion of the treatment assembly may be located in a same plane, such that an isocenter of the imaging assembly and an isocenter of the treatment assembly (substantially) coincide. In some embodiments, the treatment head may be rotatable within a first rotation plane, and a center point of the first rotation plane may be referred to as the isocenter of the treatment assembly. In some embodiments, the at least one imaging source may be rotatable within a second rotation plane, and a center point of the second rotation plane may be referred to as the isocenter of the imaging assembly. In some embodiments, the first rotation plane and/or the second rotation plane may be perpendicular to an axial direction of the first gantry or an axial direction of the second gantry.

In some embodiments, each of the at least one imaging beam may cover an imaging region. The treatment beam may cover a treatment region. The at least one imaging source and the treatment head may be configured such that the treatment region and the at least one imaging region may at least partially overlap. In some embodiments, the target volume (e.g., a region to be treated) of the subject may be placed in an overlapping region of the treatment region and the at least one imaging region.

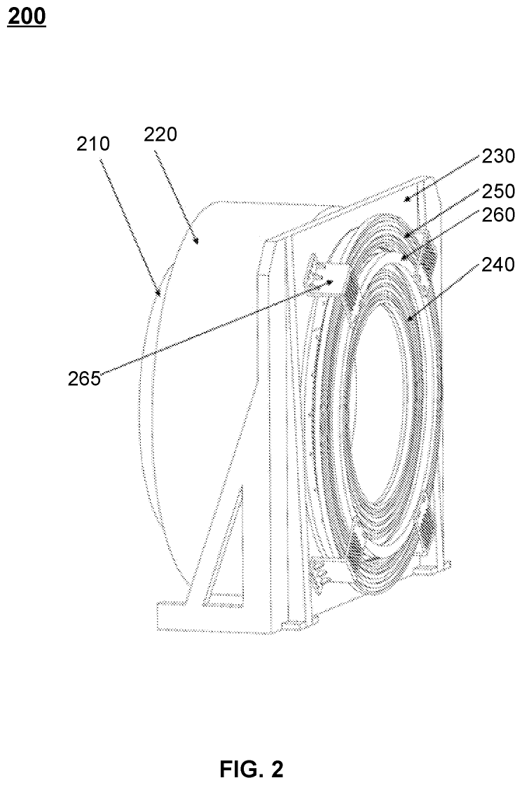

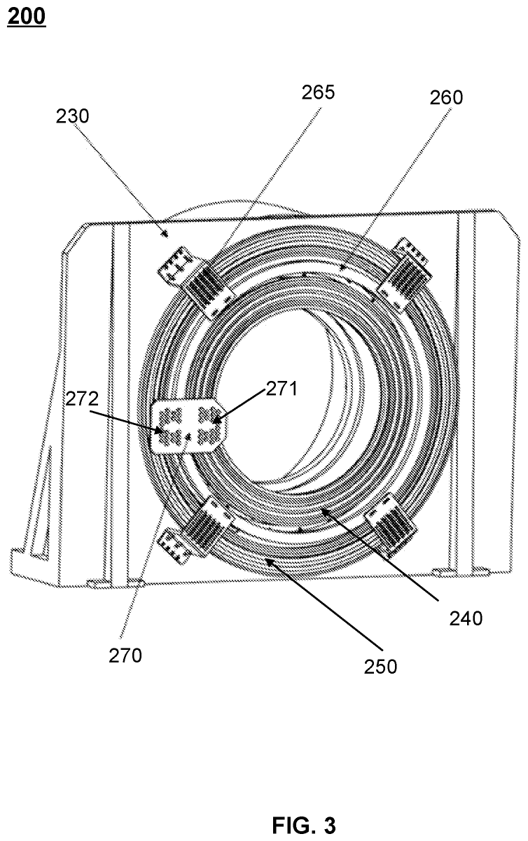

In some embodiments, at least a portion of the first gantry may be housed in the second gantry. For example, a portion of the first gantry may be housed in the second gantry. As another example, the entire first gantry may be housed in the second gantry. In some embodiments, the second gantry may include an annular structure, and at least a portion of the first gantry may be housed in the hollow space of the annular structure of the second gantry. See, e.g., FIG. 2. and FIG. 4.

In some embodiments, the first gantry may be rotatably connected to the second gantry. The second gantry may be rotatably connected to the fixed gantry. In some embodiments, the first gantry may be rotatable along a first axis (e.g., dashed line A in FIG. 4). At least one component located on the first gantry may be rotatable with the first gantry. For example, the at least one imaging source and the at least one imaging detector may be rotatable with the first gantry. The second gantry may be rotatable along a second axis (e.g., dashed line B in FIG. 4). At least one component located on the second gantry may be rotatable with the second gantry. For example, at least a portion of the treatment head (e.g., the treatment source) may be rotatable with the second gantry. In some embodiments, each of the first gantry and the second gantry may be an annular structure, respectively. The first axis or the second axis may be an axial direction of the annular structure.

In some embodiments, a radius of the second gantry may be larger than a radius of the first gantry. It should be noted “a radius” of an annular structure (e.g., a gantry, a ring (e.g., a slip ring, a static ring)) in the present disclosure may refer to an inner radius of the annular structure or an outer radius of the annular structure.

In some embodiments, the first axis may intersect or (substantially) coincide with the second axis. For example, the intersection of the first axis and the second axis may (substantially) coincide with an isocenter (e.g., an intersection of rotation axes of the first gantry or the second gantry, the treatment assembly, and the imaging assembly) of the medical device 110. As used herein, “substantially,” when used to describe a feature, indicates that a deviation from the feature is below a threshold. For instance, that the intersection of the first axis and the second axis substantially coincides with an isocenter may indicate that the distance between the isocenter of the medical device 110 and the intersection between the first axis and the second axis is below a threshold, e.g., 1 millimeter, 2 millimeters, 3 millimeters, etc. In some embodiments, the first axis may be (substantially) parallel to the second axis. As used herein, “substantially” indicates that an included angle between the first axis and the second axis is below a threshold, e.g., 1 degree, 5 degrees, 10 degrees, etc. In such cases, the imaging quality and the treatment quality may be ensured. Besides, imaging and treatment of the target volume of the subject may be performed at a position, thereby obviating the need to move the subject or the bed that supports the subject during the imaging and/or treatment and obviating the need to perform position adjustments with respect to a treatment plan of the subject.

In some embodiments, the medical device 110 may include a first bearing and a second bearing that is different from the first bearing. The first gantry may be operably connected to the second gantry through the first bearing. The second gantry may be operably connected to the fixed gantry through the second bearing. In some embodiments, a fixed portion (e.g., a stator) of the first bearing may be affixed on the second gantry, and a rotation portion (e.g., a rotor) of the first bearing may be affixed on the first gantry. A fixed portion (e.g., a stator) of the second bearing may be affixed on the fixed gantry, and a rotation portion (e.g., a rotor) of the second bearing may be affixed on the second gantry.

In some embodiments, the first bearing or the second bearing may be an electromagnetic bearing, a mechanical bearing, etc. For example, the mechanical bearing may include a slide bearing, a rolling bearing, etc. An electromagnetic bearing may be suitable for use between components undergoing high-speed rotation relative to each other. By utilizing an electromagnetic bearing, there may be no mechanical contact (and therefore no friction) between the first gantry and the second gantry, or between the second gantry and the fixed gantry, thereby extending the service life of the gantries.

In some embodiments, the medical device 110 may include a first drive component and a first transmission component. In some embodiments, the first drive component may include an electric machine, an engine, etc. The first transmission component may include a belt-drive transmission assembly, a chain-drive transmission assembly, a gear-drive transmission assembly, etc. In some embodiments, the first drive component may drive the rotation portion of the first bearing through the first transmission component, thereby driving the first gantry and the first slip ring to rotate with respect to the fixed gantry. In some embodiments, the first drive component may directly drive the rotation portion of the first bearing to rotate, thereby obviating the need to involve the first transmission component. For example, the first drive component may include a direct drive rotary (DDR) motor operably connected to the first bearing. As another example, the first drive component may include a servo motor, a stepper motor, etc.

In some embodiments, the medical device 110 may include a second drive component that is different from the first drive component and a second transmission component that is different from the first transmission component. In some embodiments, the second drive component may drive the rotation portion of the second bearing through the second transmission component, thereby driving the second gantry and the second slip ring to rotate with respect to the fixed gantry. In some embodiments, the second drive component may directly drive the rotation portion of the second bearing to rotate, thereby obviating the need to involve the second transmission component. It should be noted that the structures of the second drive component and the second transmission component may be the same as or similar to the structures of the first drive component and the first transmission component, respectively, the descriptions of which are not repeated.

In some embodiments, the first gantry may be configured to rotate independently from the second gantry. In some embodiments, the first gantry and the second gantry may be configured to rotate synchronously. In some embodiments, the medical device 110 may include a lock mechanism. In some embodiments, the lock mechanism may be located between the first gantry and the second gantry. In some embodiments, the lock mechanism may be located on the first gantry. In some embodiments, the lock mechanism may be located on the second gantry. In some embodiments, the lock mechanism may be located on both the first gantry and the second gantry.

In some embodiments, the lock mechanism may be configured to lock or unlock the first gantry and the second gantry. When the first gantry and the second gantry are locked, the first gantry and the second gantry may rotate synchronously. In some embodiments, the first gantry and the second gantry may rotate synchronously with respect to the fixed gantry. When the first gantry and the second gantry are unlocked, the first gantry may rotate independently from the second gantry. For example, the first gantry may rotate while the second gantry is stationary with respect to the fixed gantry. As another example, the second gantry may rotate while the first gantry is stationary with respect to the fixed gantry.

In some embodiments, the first drive component may be configured to drive the first transmission component to move the first bearing, and/or the second drive component may be configured to drive the second transmission component to move the second bearing in response to that the first gantry and the second gantry are locked through the lock mechanism, such that the second gantry, the second slip ring, the first gantry, and the first slip ring may rotate synchronously with respect to the fixed gantry.

In some embodiments, the lock mechanism may include an electromagnetic brake device. The electromagnetic brake device may include an electromagnetic bearing (e.g., the first bearing) and a drive component (e.g., an electric machine, an engine). The drive component may be configured to lock or unlock a rotation portion and a fixed portion of the electromagnetic bearing, such that the first gantry and the second gantry are locked or unlocked. In such cases, a transmission component may be unnecessary for locking or unlocking the first gantry and the second gantry.

In some embodiments, the lock mechanism may include at least one lock hole and at least one lock tongue. The at least one lock hole may be located on one of the first gantry and the second gantry, and the at least one lock tongue may be located on the other of the first gantry and the second gantry. In some embodiments, the at least one lock hole may be circumferentially positioned or distributed on a surface of the first gantry. The at least one lock tongue may be correspondingly positioned or distributed on a surface of the second gantry so that the at least one lock tongue may be inserted into the at least one lock hole to lock the first gantry and the second gantry. For instance, the locking mechanism may include multiple lock holes circumferentially distributed on a surface of the first gantry and multiple lock tongues circumferentially distributed on a surface of the second gantry, and each of the multiple lock tongues may be inserted to one of the multiple lock holes such that the first gantry and the second gantry are locked at different positions. As another example, each of the multiple lock tongues may be inserted to different lock holes of the multiple lock holes such that a position of the first gantry relative to the second gantry may be different.

In some embodiments, before the treatment head is caused to emit a treatment beam towards a region (e.g., a region to be treated) of a subject to perform a radiation treatment of the subject, the first gantry and the second gantry may be in an unlocked state. The first gantry may rotate independently from the second gantry to perform an imaging of the subject, or a portion thereof, e.g., the target volume of the subject. In such cases, the first gantry may rotate without the influence of the second gantry at a relatively high speed (e.g., 120 r/min), thereby completing the imaging within a short time, reducing imaging artifacts, and/or improving imaging quality. In some embodiments, after the imaging of the subject is completed, the first gantry and the second gantry may be locked through the locking mechanism. The treatment head may then be caused to emit a treatment beam to treat the target volume of the subject. The first gantry and the imaging assembly, or a portion thereof, affixed on the first gantry, may be configured with an opening through which the treatment beam may pass before traveling toward the subject. In some embodiments, after the imaging of the subject is completed, the first gantry may continue rotating independently from the second gantry, that is, during the radiation treatment, the first gantry rotates with respect the second gantry. The subject may be further imaged during the radiation treatment, and the imaging result may be used to monitor the radiation treatment.

In some embodiments, the medical device 110 may include a patient support 113. The patient support 113 may be configured to support the subject. The patient support 113 may have multiple (e.g., 6) degrees of freedom, for example, three translational degrees of freedom along three coordinate directions (i.e., x-direction, y-direction, and z-direction) and three rotational degrees of freedom around the three coordinate directions. Accordingly, the patient support 113 may move the subject along a direction of the 3D coordinate system. Merely by way of example, the patient support 113 may move the subject into the FOV of the medical device 110 along the y-direction in FIG. 1.

In some embodiments, the subject may be biological or non-biological. Merely by way of example, the subject may include a patient, a man-made subject, etc. As another example, the subject may include a specific portion, organ, and/or tissue of the patient. For example, the subject may include head, brain, neck, body, shoulder, arm, thorax, cardiac, stomach, blood vessel, soft tissue, knee, feet, or the like, or any combination thereof. In the present disclosure, “subject” and “object” are used interchangeably.

It should be noted that the above descriptions of the medical device 110 are for illustration purposes and are non-limiting. In some embodiments, the first medical assembly (e.g., the imaging assembly, the treatment assembly) and the second medical assembly (e.g., the imaging assembly, the treatment assembly) may be arranged, e.g., along the y-axis in FIG. 1. The subject may be moved to the imaging region or the treatment region by moving the patient support 113. In some embodiments, the medical device 110 may include only the imaging assembly, which performs the IGRT in combination with an external treatment assembly that is not part of the medical device 110. In some embodiments, the medical device 110 may include only the treatment assembly, which performs the IGRT in combination with an external imaging assembly that is not part of the medical device 110.

The network 150 may facilitate exchange of information and/or data. In some embodiments, one or more components of the medical system 100 (e.g., the medical device 110, the processing device 120, the storage device 130, or the terminal 140) may send information and/or data to another component(s) in the medical system 100 via the network 150. For example, the processing device 120 may obtain a user instruction from the terminal 140 via the network 150. As another example, the processing device 120 may obtain scan data (e.g., projection data) from the medical device 110 via the network 150. In some embodiments, the network 150 may be any type of wired or wireless network, or combination thereof. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network), a cellular network (e.g., a Long Term Evolution (LTE) network), a frame relay network, a virtual private network (“VPN”), a satellite network, a telephone network, routers, hubs, switches, server computers, and/or any combination thereof. Merely by way of example, the network 150 may include a cable network, a wireline network, an optical fiber network, a telecommunications network, an intranet, an Internet, a local area network (LAN), a wide area network (WAN), a wireless local area network (WLAN), a metropolitan area network (MAN), a wide area network (WAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired or wireless network access points such as base stations and/or internet exchange points through which one or more components of the medical system 100 may be connected to the network 150 to exchange data and/or information.

The terminal 140 may include a mobile device 140-1, a tablet computer 140-2, a laptop computer 140-3, or the like, or any combination thereof. In some embodiments, the mobile device 140-1 may include a smart home device, a wearable device, a smart mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a bracelet, footgear, eyeglasses, a helmet, a watch, clothing, a backpack, an accessory, or the like, or any combination thereof. In some embodiments, the smart mobile device may include a smartphone, a personal digital assistant (PDA), a gaming device, a navigation device, a point of sale (POS) device, or the like, or any combination thereof. In some embodiments, the virtual reality device and/or the augmented reality device may include a virtual reality helmet, a virtual reality glass, a virtual reality patch, an augmented reality helmet, an augmented reality glass, an augmented reality patch, or the like, or any combination thereof. For example, the virtual reality device and/or the augmented reality device may include a Google Glass, an Oculus Rift, a HoloLens, a Gear VR, etc. In some embodiments, the terminal 140 may remotely operate the medical device 110. In some embodiments, the terminal 140 may operate the medical device 110 via a wireless connection. In some embodiments, the terminal 140 may receive information and/or instructions inputted by a user, and send the received information and/or instructions to the medical device 110 or to the processing device 120 via the network 150. In some embodiments, the terminal 140 may receive data and/or information from the processing device 120. In some embodiments, the terminal 140 may be part of the processing device 120. In some embodiments, the terminal 140 may be omitted.

In some embodiments, the processing device 120 may process data obtained from the medical device 110, the storage device 130, or the terminal 140. For example, the processing device 120 may obtain projection data of a subject from the medical device 110 and generate an image of the subject based on the projection data. As another example, the processing device 120 may cause one or more components (e.g., the treatment head, the at least one imaging source, the at least one imaging detector, the treatment detector, the patient support 113, the gantry assembly 111, etc.) of the medical device 110 to be located at a specific position. The processing device 120 may be a central processing unit (CPU), a digital signal processor (DSP), a system on a chip (SoC), a microcontroller unit (MCU), or the like, or any combination thereof.

In some embodiments, the processing device 120 may be a single server or a server group. The server group may be centralized or distributed. In some embodiments, the processing device 120 may be local or remote. For example, the processing device 120 may access information and/or data stored in the medical device 110, the storage device 130, and/or the terminal 140 via the network 150. As another example, the processing device 120 may be directly connected to the medical device 110, the storage device 130, and/or the terminal 140, to access stored information and/or data. In some embodiments, the processing device 120 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

The storage device 130 may store data and/or instructions. In some embodiments, the storage device 130 may store data obtained from the terminal 140 and/or the processing device 120. For example, the storage device 130 may store one or more images generated by the processing device 120. In some embodiments, the storage device 130 may store data and/or instructions that the processing device 120 may execute or use to perform exemplary methods described in the present disclosure. For example, the storage device 130 may store instructions that the processing device 120 may execute or use to generate one or more images based on projection data. In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more components of the medical system 100 (e.g., the medical device 110, the processing device 120, the terminal 140). One or more components of the medical system 100 may access the data or instructions stored in the storage device 130 via the network 150. In some embodiments, the storage device 130 may be directly connected to or communicate with one or more components of the medical system 100 (e.g., the processing device 120, the terminal 140). In some embodiments, the storage device 130 may be part of the processing device 120.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Some embodiments of the present disclosure may provide a slip ring assembly. The slip ring assembly may include a first slip ring (e.g., a conductive slip ring), a second slip ring (e.g., a conductive slip ring), and a transmission component. The transmission component may be operably connected to the first slip ring and the second slip ring, and data transmission (e.g., signal transmission) may be implemented between the first slip ring and the transmission component, and between the second slip ring and the transmission component. In some embodiments, the transmission component may be configured to transmit at least one signal of the first slip ring and at least one signal of the second slip ring. In some embodiments, power transmission (e.g., an electric current, an electric voltage, an electric power) may also be implemented between the first slip ring and the transmission component, and between the second slip ring and the transmission component.

In some embodiments, the slip ring assembly may be configured in (e.g., integrated in) the medical device 110. The first slip ring may be operably connected to the first gantry. The second slip ring may be operably connected to the second gantry. The transmission component may be operably connected to (e.g., located on) the fixed gantry. It is understood that, as used herein, when a component is referred to as being “integrated in” another component, it may be directly on, connected or coupled to, or communicate with the other component, or an intervening component may be present, unless the context clearly indicates otherwise.

In some embodiments, the slip ring assembly may be configured to facilitate data transmission of and/or supply power (e.g., an electric current, an electric voltage, a power) to at least one component (e.g., the imaging assembly, the treatment assembly) of the medical device 110. In some embodiments, the slip ring assembly may be configured to transmit data to a control component (e.g., the processing device 120, the terminal 140) of the medical system 100. The control component may be configured to control the imaging and/or the radiation treatment of the subject. In some embodiments, the slip ring assembly may be configured to receive data from the control component. For example, the data may include imaging data of the subject, e.g., a region being imaged, a time duration for the imaging, an imaging protocol, a radiation dose, raw data acquired in the imaging, etc. As another example, the data may include treatment data of the subject, e.g., a region being treated, a radiation dose, a time duration for the treatment, imaging data acquired during a treatment session (using, e.g., an EPID) etc. As a further example, the data may include input/output (I/O) data, a pulse signal, an analog signal, a digital signal, a controller area network (CAN) signal, etc. In some embodiments, the data may be transmitted according to a communication protocol, e.g., peripheral component interconnect express (PCIe), Ethernet, etc.

In some embodiments, the first slip ring may be configured to rotate independently from the second slip ring. In some embodiments, the first slip ring and the second slip ring may be configured to rotate synchronously. As described above, in some embodiments, the slip ring assembly may be configured in the medical device 110. The first slip ring may be located on and rotatable with the first gantry. The second slip ring may be located on and rotatable with the second gantry.

In some embodiments, the transmission component may include at least one of a static ring, a signal generator, or a signal receiver. The signal generator and the signal receiver may be configured to facilitate the data transmission. In some embodiments, the signal generator may be configured to generate a signal corresponding to the data to be transmitted (e.g., the at least one signal of the first slip ring, the at least one signal of the second slip ring). The signal receiver may be configured to receive a signal corresponding to the data to be transmitted (e.g., the at least one signal of the first slip ring, the at least one signal of the second slip ring). In some embodiments, the static ring may be located on (e.g., affixed on) the fixed gantry. The signal generator and the signal receiver may be located on the static ring. In some embodiments, the static ring may be affixed on the fixed gantry through a fixation mechanism (e.g., 265 in FIG. 2 and FIG. 3). The fixation mechanism may include a first fixation portion, a second fixation portion, and a connection portion. The first fixation portion may be operably connected to the static ring, the second fixation portion may be operably connected to the fixed gantry, and the connection portion may be operably connected to the first fixed portion and the second fixed portion. It should be noted that the transmission component may include more than one fixation mechanism. For instance, the transmission component may include 1, 2, 4, 7, etc., fixation mechanisms.

In some embodiments, at least two of the first slip ring, the second slip ring, or the static ring may wind around each other. For example, the first slip ring may wind around the static ring. As another example, the second slip ring may wind around the static ring. As a further example, the second slip ring may wind around the first slip ring. As a still further example, the first slip ring, the second slip ring, and the static ring may be arranged such that the static ring is located between the first slip ring and the second slip ring with the static ring located outside and winding around the first slip ring and the second slip ring located outside and winding around the static ring.

In some embodiments, at least two of the first slip ring, the second slip ring, or the static ring may be located in a same plane. As used herein, that two rings are located in a same plane may refer to that specific points (e.g., a center of gravity, a center of mass) of the two rings are located in the same plane. In some embodiments, the plane may be perpendicular to an axial direction of the slip ring assembly (e.g., parallel to the y-axis in FIG. 1). The axial direction of the slip ring assembly may be parallel to an axial direction of the first slip ring, an axial direction of the second slip ring, or an axial direction of the static ring. In some embodiments, the first slip ring and the second slip ring may be located in the same plane. The axial direction of the first slip ring may intersect, (substantially) coincide with, or be (substantially) parallel to the axial direction of the second slip ring. In some embodiments, the first slip ring, the second slip ring, the static ring may be located in the same plane. The axial direction of the first slip ring may intersect, (substantially) coincide with, or be (substantially) parallel to the axial direction of the second slip ring and the axial direction of the static ring. As used herein, “substantially” indicates that an included angle between two axial directions is below a threshold, e.g., 1 degree, 5 degrees, 10 degrees, etc.

In some embodiments, at least two of the first slip ring, the second slip ring, or the static ring may be disposed concentrically. In some embodiments, the first slip ring and the second slip ring may be disposed concentrically. A central point (e.g., a center of gravity, a center of mass) of the first slip ring may (substantially) coincide with a central point (e.g., a center of gravity, a center of mass) of the second slip ring. In some embodiments, the first slip ring, the second slip ring, and the static ring may be disposed concentrically. The central point of the first slip ring may (substantially) coincide with the central point of the second slip ring and a central point of the static ring. As used herein, “substantially” indicates that a distance between two central points is below a threshold, e.g., 1 millimeter, 2 millimeters, 3 millimeters, etc. In some embodiments, as described above, the slip ring assembly may be configured in the medical device 110.

In some embodiments, the static ring may be located between the first slip ring and the second slip ring. In some embodiments, a radius of the static ring may be larger than a radius of the first slip ring, and a radius of the second slip ring may be larger than the radius of the static ring, such that the static ring is located between the first slip ring and the second slip ring. In some embodiments, a difference between the radius of the static ring and the radius of the first slip ring may be smaller than a first difference threshold. In some embodiments, a difference between the radius of the static ring and the radius of the second slip ring may be smaller than a second difference threshold. In some embodiments, the radius of the static ring may be smaller than a radius threshold (e.g., 1.7 meters, 1.6 meters, 1.5 meters) in order to achieve reliable data transmission involving the first slip ring and/or the second slip ring that is facilitated by the static ring.

In some embodiments, at least two of the first slip ring, the second slip ring, or the static ring may be arranged along an axial direction of the slip ring assembly (e.g., parallel to the y-axis in FIG. 1). The axial direction of the slip ring assembly may be parallel to an axial direction of the first slip ring, an axial direction of the second slip ring, and/or an axial direction of the static ring. For example, the first slip ring and the second slip ring may be arranged (coaxially) along the axial direction of the slip ring assembly. As another example, the first slip ring and the static ring may be arranged (coaxially) along the axial direction of the slip ring assembly. As a further example, the second slip ring and the static ring may be arranged (coaxially) along the axial direction of the slip ring assembly.

In some embodiments, the slip ring assembly may include at least one carbon brush assembly. The at least one carbon brush assembly may contact at least one component of the slip ring assembly. In some embodiments, the at least one carbon brush assembly may be configured to supply power to and/or facilitate data transmission of the first slip ring and/or the second slip ring. In some embodiments, the at least one carbon brush assembly may be affixed on a fixed portion of the slip ring assembly (e.g., the static ring), a fixed portion of the medical device 110 (e.g., the fixed gantry), etc.

In some embodiments, the at least one carbon brush assembly may include a first carbon brush assembly operably connected to (e.g., in contact with) the first slip ring and a second carbon brush assembly operably connected to (e.g., in contact with) the second slip ring. The first carbon brush assembly may be configured to facilitate first data transmission (also referred to as first contact data transmission) and/or first power transmission between the first slip ring and the transmission component. The second carbon brush assembly may be configured to facilitate second data transmission (also referred to as second contact data transmission) and/or second power transmission between the second slip ring and the transmission component. In some embodiments, the first carbon brush assembly may include multiple carbon brushes that are in contact with the first slip ring. The second carbon brush assembly may include multiple carbon brushes that are in contact with the second slip ring. In some embodiments, the first carbon brush assembly and the second carbon brush assembly may be two separated assemblies that lack data communication between each other. In some embodiments, the first carbon brush assembly and the second carbon brush assembly may be an integrated assembly, thereby facilitating the installation, the maintenance, and/or the replacement of the carbon brush assembly. It should be noted that the above descriptions are for illustration purposes and non-limiting. In some embodiments, the slip ring assembly may include only one carbon brush assembly. The carbon brush assembly may be configured to facilitate the first data transmission and the second data transmission.

In some embodiments, the first slip ring may include at least one first channel (e.g., at least one copper ring) configured to facilitate the first data transmission between the first slip ring and the static ring. Each of the at least one first channel may be operably connected to one or more carbon brushes of the first carbon brush assembly. In some embodiments, the at least one first channel may also be configured to facilitate the first power transmission between the first slip ring and the static ring. In some embodiments, different first channels of the at least one first channel may have different transmission capabilities. The larger a transmission capability of a first channel, the more carbon brushes that are operably connected to the first channel may be.

In some embodiments, one or more of the at least one first channel may be selected for the first data transmission and/or the first power transmission according to practical demands. In some embodiments, a first channel of a relatively large transmission capability may be used to implement the first power transmission. A first channel of a relatively small transmission capability may be used to facilitate the first data transmission, for example, an I/O signal, an analog signal, a CAN signal, a digital signal, etc.

In some embodiments, similar to the first slip ring, the second slip ring may include at least one second channel configured to facilitate the second data transmission between the second slip ring and the static ring. In some embodiments, the at least one second channel may also be configured to facilitate the second power transmission between the second slip ring and the static ring. Each of the at least one second channel may be operably connected to one or more carbon brushes of the second carbon brush assembly. In some embodiments, different second channels of the at least one second channel may have different transmission capabilities. The larger a transmission capability of a second channel, the more carbon brushes that are operably connected to the second channel may be.

In some embodiments, one or more of the at least one second channel may be selected for the second data transmission and/or the second power transmission according to practical demands. In some embodiments, a second channel of a relatively large transmission capability may be used to implement the second power transmission. A second channel of a relatively small transmission capability may be used to implement the second data transmission, for example, an I/O signal, an analog signal, a CAN signal, a digital signal, etc.

In some embodiments, the transmission component may include at least one transmission module and at least one receiving module that are configured to facilitate first contactless data transmission between the first slip ring and the transmission component or second contactless data transmission between the second slip ring and the transmission component. In some embodiments, the first contactless data transmission or the second contactless data transmission may include multi-channel data transmission.

In some embodiments, the at least one transmission module may include a first transmission module and a second transmission module. The at least one receiving module may include a first receiving module and a second receiving module. The first transmission module and the first receiving module may be configured to facilitate the first contactless data transmission between the first slip ring and the transmission component. The second transmission module and the second receiving module may be configured to facilitate the second contactless data transmission between the second slip ring and the transmission component.

In some embodiments, the at least one transmission module may also include a third transmission module and a fourth transmission module that are different from the first transmission module and the second transmission module, respectively. The at least one receiving module may also include a third receiving module and a fourth receiving module that are different from the first receiving module and the second receiving module, respectively. The third transmission module and the third receiving module may also be configured to facilitate the first contactless data transmission between the first slip ring and the transmission component. The fourth transmission module and the fourth receiving module may also be configured to facilitate the second contactless data transmission between the second slip ring and the transmission component. Thus, two-channel first data transmission and two-channel second data transmission may be achieved, respectively.

In some embodiments, the at least one transmission module may be located on the first slip ring or the second slip ring, and the at least one receiving module may be located on the static ring, such that data is transmitted from the first slip ring or the second slip ring to the static ring. In some embodiments, the at least one transmission module may be located on the static ring, and the at least one receiving module may be located on the first slip ring or the second slip ring, such that data may be transmitted from the static ring to the first slip ring or the second slip ring. In such cases, a direction of the data transmission may be adjusted by adjusting positions of the at least one transmission module and the at least one receiving module. For example, after the medical device 110 is assembled, the direction of the data transmission may be adjusted by interchanging the mounting positions of the at least one transmission module and the mounting positions the at least one receiving module.

It should be noted the above descriptions are for illustration purposes and non-limiting. In some embodiments, a count of the transmission modules may be non-limiting, e.g., 1, 3, 4, etc. A count of the receiving modules may be non-limiting, e.g., 1, 3, 4, etc. In some embodiments, the count of the transmission modules may be the same as the count of the receiving modules.

In some embodiments, each of the at least one transmission module may include a transmitter and an antenna. The transmitter may be configured to transmit data to a corresponding receiving module through the antenna. In some embodiments, the corresponding receiving module may further transmit the data to the control component of the medical system 100 (e.g., the processing device 120, the terminal 140), the first slip ring, or the second slip ring. For example, the corresponding receiving module may first transmit data of the first slip ring to the control component and then to the second slip ring. As another example, the corresponding receiving module may directly transmit the data of the first slip ring to the second slip ring. As a further example, the corresponding receiving module may first transmit data of the second slip ring to the control component and then to the first slip ring. As still a further example, the corresponding receiving module may directly transmit the data of the second slip ring to the first slip ring.

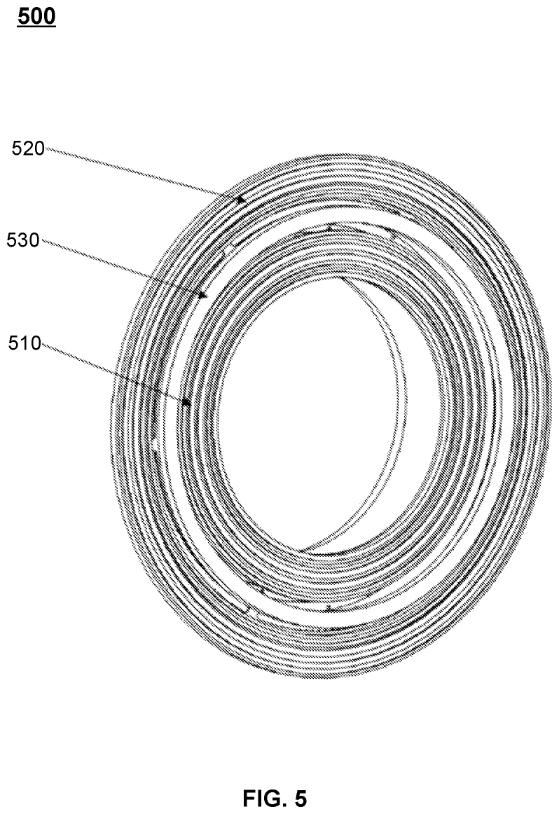

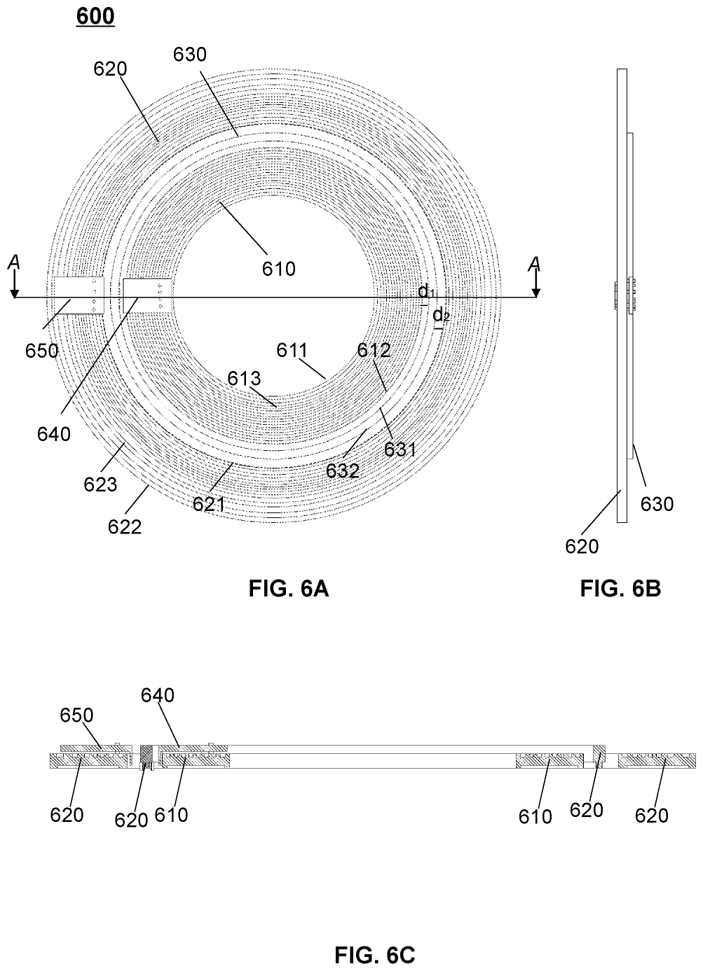

In some embodiments, the transmission module may be opposingly positioned with respect to the corresponding receiving module. In some embodiments, the transmission module may be located on an outer sidewall (e.g., 612 in FIG. 6A) of the first slip ring that is closer to an inner sidewall (e.g., 631 in FIG. 6A) of the static ring than an inner sidewall (e.g., 611 in FIG. 6A) of the first slip ring. The corresponding receiving module may be located on the inner sidewall of the static ring. In such cases, a distance between the transmission module (e.g., a surface thereof) and the corresponding receiving module (e.g., a surface thereof) may be smaller than a first distance threshold (e.g., 1 millimeter, 2 millimeters, 3 millimeters). For example, the distance may be equal to a distance (e.g., d₁ in FIG. 6A) between the outer sidewall of the first slip ring and the inner sidewall of the static ring.

In some embodiments, the transmission module may be located on an inner sidewall (e.g., 621 in FIG. 6A) of the second slip ring that is closer to an outer sidewall (e.g., 632 in FIG. 6A) of the static ring than an outer sidewall (e.g., 622 in FIG. 6A) of the second slip ring. The second receiving module may be located on the outer sidewall of the static ring. In such cases, a distance between the transmission module (e.g., a surface thereof) and the receiving module (e.g., a surface thereof) may be smaller than a second distance threshold (e.g., 1 millimeter, 2 millimeters, 3 millimeters). For example, the distance may be equal to a distance (e.g., d₂ in FIG. 6A) between the inner sidewall of the second slip ring and the outer sidewall of the static ring. In some embodiments, the first distance threshold may be the same as or different from the second distance threshold.

In some embodiments, a speed of the first contactless data transmission and/or the second contactless data transmission may be higher than the speed of the first contact data transmission and/or the second contact data transmission. In some embodiments, the speed of the first contactless data transmission and/or the second contactless data transmission may exceed a speed threshold, for example, 2.5 Gbps, 5 Gbps, 6 Gbps, 6.25 Gbps, 8 Gbps, 8.5 Gbps, 10 Gbps, 16 Gbps, 32 Gbps etc. In some embodiments, the first contactless data transmission and/or the second contactless data transmission may be implemented according to a communication protocol, e.g., peripheral component interconnect express (PCIe), etc. In some embodiments, at least two of the first contact data transmission, the second contact data transmission, the first contactless data transmission, or the second contactless data transmission may procced simultaneously or alternately.

In some embodiments, the slip ring assembly may include a first slip ring, a second slip ring, a first transmission component, and a second transmission component. The first transmission component may be operably connected to the first slip ring. The second transmission component may be operably connected to the second slip ring. In some embodiments, the first transmission component may be configured to transmit at least one signal of the first slip ring. The second transmission component may be configured to transmit at least one signal of the second slip ring.

In some embodiments, the first transmission component may include a first static ring, and the second transmission component may include a second static ring. In some embodiments, a radius of the first static ring and/or a radius of the second static ring may be smaller than or equal to a radius of the first slip ring and/or a radius of the second slip ring, such that the radius of the first static ring and/or the radius of the second static ring is smaller than a threshold (e.g., 1.7 meters, 1.6 meters). For example, the radius of the first static ring may be smaller than or equal to the radius of the first slip ring. As another example, the radius of the first static ring may be smaller than or equal to the radius of the second slip ring. As a further example, the radius of the second static ring may be smaller than or equal to the radius of the first slip ring. As still a further example, the radius of the second static ring may be smaller than or equal to the radius of the second slip ring.

In some embodiments, a radius of the first static ring and/or a radius of the second static ring may be larger than the radius of the first slip ring and/or the radius of the second slip ring. For example, the radius of the first static ring may be larger than the radius of the first slip ring. As another example, the radius of the first static ring may be larger than the radius of the second slip ring. As a further example, the radius of the second static ring may be larger than the radius of the first slip ring. As still a further example, the radius of the second static ring may be larger than the radius of the second slip ring.

In some embodiments, the first transmission component (e.g., the first static ring) and the second transmission component (e.g., the second static ring) may be located on the two sides of the first slip ring along an axial direction of the first slip ring. In some embodiments, the first transmission component (e.g., the first static ring) and the second transmission component (e.g., the second static ring) may be located on the two sides of the second slip ring along an axial direction of the second slip ring. In some embodiments, the first transmission component (e.g., the first static ring) and the second transmission component (e.g., the second static ring) may be located on a side of the first slip ring and/or the second slip ring that is further away from the first gantry, e.g., along the negative y-axis in FIG. 1. In some embodiments, the first transmission component (e.g., the first static ring) and the second transmission component (e.g., the second static ring) may be located on a side of the first slip ring and/or the second slip ring that is closer to the first gantry, e.g., along the negative y-axis in FIG. 1.

In some embodiments, at least two of the first slip ring, the second slip ring, the first transmission component (e.g., the first static ring), or the second transmission component (e.g., the second static ring) may be disposed coaxially, e.g., along the y-axis in FIG. 1. For example, the first slip ring and the second slip ring may be disposed coaxially. As another example, the first slip ring, the second slip ring, and the first static ring may be disposed coaxially. As a further example, the first slip ring, the second slip ring, the first static ring, and the second static ring may be disposed coaxially.

In some embodiments, the slip ring assembly may include at least one carbon brush assembly. The at least one carbon brush assembly may be operably connected to the first slip ring and the second slip ring. The at least one carbon brush assembly may be configured to facilitate first data transmission between the first slip ring and the transmission component and second data transmission between the second slip ring and the transmission component. In some embodiments, the at least one carbon brush assembly may be affixed on a fixed portion of the slip ring assembly (e.g., the static ring), a fixed portion of the medical device 110 (e.g., the fixed gantry), etc.

In some embodiments, the first slip ring assembly may include a first carbon brush assembly operably connected to (e.g., in contact with) the first slip ring and a second carbon brush assembly operably connected to (e.g., in contact with) the second slip ring. The first carbon brush assembly may be configured to facilitate the first data transmission, and the second carbon brush assembly may be configured to facilitate the second data transmission. In some embodiments, the slip ring assembly may include only one carbon brush assembly. The carbon brush assembly may be configured to facilitate the first data transmission and the second data transmission. In some embodiments, the carbon brush assemblies described above may also be configured to facilitate first power transmission between the first slip ring and the transmission component and/or second power transmission between the second slip ring and the transmission component. In some embodiments, the first transmission component and/or the second transmission component may be the same as or similar to the transmission component, more descriptions of which are not repeated.

In some embodiments, the slip ring assembly as illustrated above may be configured in (e.g., integrated in) the medical device 110. The first slip ring may be operably connected to the first gantry. The second slip ring may be operably connected to the second gantry. The first transmission component and the second transmission component may be operably connected to (e.g., located on) the fixed gantry. In some embodiments, the first transmission component and/or the first slip ring may be located on one side of the fixed gantry, and the second transmission component and/or the second slip ring may be located on another side of the fixed gantry along an axial direction of the fixed gantry. In some embodiments, the first transmission component and/or the first slip ring may be located on one side of the first gantry or the second gantry, and the second transmission component and/or the second slip ring may be located on another side of the first gantry or the second gantry along an axial direction of the first gantry or along an axial direction of the second gantry. For example, the first transmission component and the first slip ring may be located on one side of the first gantry, and the second transmission component and the second slip ring may be located on another side of the first gantry along the axial direction of the first gantry. As another example, the first transmission component and the first slip ring may be located on one side of the second gantry, and the second transmission component and the second slip ring may be located on another side of the second gantry along the axial direction of the second gantry. In some embodiments, the first transmission component, the first slip ring, the second transmission component, and the second slip ring may be located on a same side of the first gantry or the second gantry along the axial direction of the first gantry or along the axial direction of the second gantry.

In some embodiments, the transmission component may include at least one transmission module and at least one receiving module that are configured to facilitate first contactless data transmission between the first slip ring and the transmission component. In some embodiments, the first contactless data transmission may include multi-channel data transmission.

In some embodiments, the at least one transmission module may include a first transmission module and a second transmission module. The at least one receiving module may include a first receiving module and a second receiving module. The first transmission module and the first receiving module may be configured to facilitate the first contactless data transmission between the first slip ring and the transmission component, and the second transmission module and the second receiving module may also be configured to facilitate the first contactless data transmission between the first slip ring and the transmission component, thereby achieving two-channel data transmission.

In some embodiments, the second transmission component may include at least one transmission module and at least one receiving module that are configured to facilitate second contactless data transmission between the second slip ring and the transmission component. In some embodiments, the second contactless data transmission may include multi-channel data transmission.

In some embodiments, the at least one transmission module may include a first transmission module and a second transmission module. The at least one receiving module may include a first receiving module and a second receiving module. The first transmission module and the first receiving module may be configured to facilitate the second contactless data transmission between the second slip ring and the second transmission component. The second transmission module and the second receiving module may also be configured to facilitate the second contactless data transmission between the second slip ring and the second transmission component, thereby achieving two-channel data transmission. More descriptions of the first contactless data transmission and/or the second contactless data transmission may be found in the description of the embodiments in which the first slip ring and the second slip ring share the transmission component as illustrated above, and not repeated here.

According to some embodiments of the present disclosure, a method for operating a system (e.g., the medical system 100) may be provided. The system may include a first medical assembly, a second medical assembly, and a slip ring assembly. The slip ring assembly may be configured to facilitate data transmission of the first medical assembly, data transmission of the second medical assembly, power transmission of the first slip ring assembly, or power transmission of the second slip ring assembly. The method may include: obtaining data of a first portion of the first medical assembly or a first portion of the second medical assembly; and transmitting, through the slip ring assembly, the data to a second portion of the first medical assembly, a second portion of the second medical assembly, or a control component of the system.

In some embodiments, the data may include imaging data of a subject or treatment data of the subject acquired by the first medical assembly or the second medical assembly.

In some embodiments, the transmitting, through the slip ring assembly, the data to a second portion of the first medical assembly, a second portion of the second medical assembly, or a control component of the system may include: transmitting the data through a contact data transmission mode.

In some embodiments, the transmitting, through the slip ring assembly, the data to a second portion of the first medical assembly, a second portion of the second medical assembly, or a control component of the system may include: transmitting the data through a contactless data transmission mode.

FIG. 2 and FIG. 3 are schematic diagrams illustrating an exemplary medical device according to some embodiments of the present disclosure. The medical device 200 may be an example of the medical device 110 in FIG. 1.

As shown in FIG. 2 and FIG. 3, the medical device 200 may include a first gantry 210, a second gantry 220, a fixed gantry 230, and a slip ring assembly. The slip ring assembly may include a first slip ring 240, a second slip ring 250, and a transmission component 260. The first gantry 210 may be configured to accommodate at least a first portion of a first medical assembly (e.g., an imaging assembly, a treatment assembly) and/or at least a first portion of a second medical assembly (e.g., an imaging assembly, a treatment assembly). The second gantry 220 may be configured to accommodate at least a second portion of the first medical assembly and at least a second portion of the second medical assembly. The fixed gantry 230 may be configured to support the first gantry 210 and the second gantry 220. As shown in FIG. 2, at least a portion of the first gantry 210 may be housed in the second gantry 220, and the space occupied by the medical device 200 (e.g., along the y-axis in FIG. 1) may be reduced.

In some embodiments, the first slip ring 240 may be operably connected to the first gantry 210. The first slip ring 240 may be located on the first gantry 210. In some embodiments, the first gantry 210 may be rotatable. The first slip ring 240 may be rotatable with the first gantry 210. In some embodiments, the second slip ring 250 may be operably connected to the second gantry 220. The second slip ring 250 may be located on the second gantry 220. In some embodiments, the second gantry 220 may be rotatable. The second slip ring 250 may be rotatable with the second gantry 220.

In some embodiments, the transmission component 260 may be operably connected to the fixed gantry 230. The transmission component 260 may be located on the fixed gantry 230. As shown in FIG. 2, the medical device 200 may include four fixation mechanisms 265. The fixation mechanisms 265 may be configured to affix the transmission component 260 on the fixed gantry 230. In some embodiments, one of the fixation mechanisms 265 may include a first fixed portion, a second fixed portion, and a connection portion (not shown in FIG. 2 and FIG. 3). The first fixed portion may be operably connected to the transmission component 260. The second fixed portion may be operably connected to the fixed gantry 230. The connection portion may be operably connected to both the first fixed portion and the second fixed portion.

In some embodiments, the first slip ring 240 may be configured to supply power to and/or facilitate data transmission of at least one component (e.g., the first portion of the first medical assembly, the first portion of the second medical assembly) located on the first gantry 210. For example, data to be transmitted may include imaging data of a subject, treatment data of the subject, etc. In some embodiments, the second slip ring 250 may be configured to supply power to and/or facilitate data transmission of at least one component (e.g., the second portion of the first medical assembly, the second portion of the second medical assembly) located on the second gantry 220. For example, data to be transmitted may include imaging data of a subject, treatment data of the subject, etc.

As shown in FIG. 2 and FIG. 3, the transmission component 260 may include a static ring. The static ring may be located between the first slip ring 240 and the second slip ring 250. A radius of the static ring may be larger than the first slip ring 240, and a radius of the second slip ring 250 may be larger than the static ring. In some embodiments, the first slip ring 240, the second slip ring 250, and the static ring may be located in a same plane.

In some embodiments, as shown in FIG. 3, the medical device 200 may include a carbon brush assembly 270. The carbon brush assembly 270 may be configured to supply power to and/or facilitate data transmission of at least one component (e.g., the first slip ring 240, the second slip ring 250, the imaging assembly, the treatment assembly) located on the first gantry 210 and/or the second gantry 220. In some embodiments, the carbon brush assembly 270 may include at least one first carbon brush 271 operably connected to (e.g., in contact with) the first slip ring 240 and at least one second carbon brush 272 operably connected to (e.g., in contact with) the second slip ring 250. The first carbon brush(es) 271 may be configured to facilitate first data transmission and/or first power transmission between the first slip ring 240 and the transmission component 260. The second carbon brush(es) 272 may be configured to facilitate second data transmission and/or second power transmission between the second slip ring 250 and the transmission component 260. As shown in FIG. 3, the first carbon brush(es) 271 and the second carbon brush(es) 272 may be integrated as a single piece. More descriptions of the medical device 200 may be found elsewhere in the present disclosure, for example, the descriptions of the medical device 110 in FIG. 1.

FIG. 4 is a section view illustrating an exemplary medical device according to some embodiments of the present disclosure. The medical device 400 may be an example of the medical device 110 in FIG. 1 or the medical device 200 in FIG. 2 and FIG. 3.

As shown in FIG. 4, the medical device 400 may include a first gantry 410, a second gantry 420, a fixed gantry 430, and a slip ring assembly. The slip ring assembly may include a first slip ring 440, a second slip ring 450, and a transmission component 460. The first gantry 410 may be configured to accommodate at least a first portion of a first medical assembly (e.g., an imaging assembly, a treatment assembly) and at least a first portion of a second medical assembly (e.g., an imaging assembly, a treatment assembly). The second gantry 420 may be configured to accommodate at least a second portion of the first medical assembly and at least a second portion of the second medical assembly. The fixed gantry 430 may be configured to support the first gantry 410 and the second gantry 420. As shown in FIG. 4, at least a portion of the first gantry 410 may be housed in the second gantry 420.

As shown in FIG. 4, the medical device 400 may include a bearing 470 and a bearing 480. The first gantry 410 may be operably connected to the second gantry 420 through the bearing 470. The second gantry 420 may be operably connected to the fixed gantry 430 through the bearing 480.

In some embodiments, the first slip ring 440 may be operably connected to the first gantry 410. The first slip ring 440 may be located on the first gantry 410. In some embodiments, the first gantry 410 may be rotatable, e.g., along an axial direction (e.g., the dashed line A) of the first gantry 410. The first slip ring 440 may be rotatable with the first gantry 410. In some embodiments, the second slip ring 450 may be operably connected to the second gantry 420. The second slip ring 250 may be located on the second gantry 420. In some embodiments, the second gantry 420 may be rotatable, e.g., along an axial direction (e.g., the dashed line B) of the second gantry 420. The second slip ring 450 may be rotatable with the second gantry 420. In some embodiments, the transmission component 460 may be operably connected to the fixed gantry 430. The transmission component 460 may be located on the fixed gantry 430.

In some embodiments, the first slip ring 440 may be configured to supply power to and/or facilitate data transmission of at least one component (e.g., the first portion of the imaging assembly, the first portion of the treatment assembly) located on the first gantry 410. For example, data to be transmitted may include imaging data of a subject, treatment data of the subject, etc. In some embodiments, the second slip ring 450 may be configured to supply power to and/or facilitate data transmission of at least one component (e.g., the second portion of the imaging assembly, the second portion of the treatment assembly) located on the second gantry 420. For example, data to be transmitted may include imaging data of a subject, treatment data of the subject, etc.

In some embodiments, the first gantry 410 may be configured to rotate independently from the second gantry 420. In some embodiments, the first gantry 410 and the second gantry 420 may be configured to rotate synchronously. As shown in FIG. 4, the medical device 400 may include a lock mechanism 490. In some embodiments, the first gantry 410 and the second gantry 420 may be locked through the lock mechanism 490, and the first gantry and the second gantry may rotate synchronously. In some embodiments, the first gantry and the second gantry may be unlocked, and the first gantry 410 may rotate independently from the second gantry 420. More descriptions of the medical device 400 may be found elsewhere in the present disclosure, for example, the descriptions of the medical device 110 in FIG. 1 or the medical device 200 in FIG. 2 and FIG. 3.

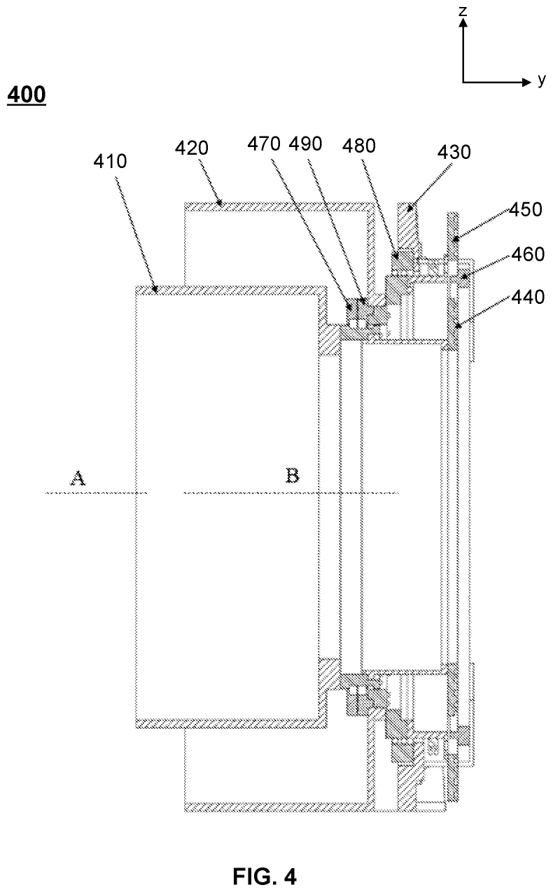

FIG. 5 is a schematic diagram illustrating an exemplary slip ring assembly according to some embodiments of the present disclosure. The slip ring assembly 500 may be an example of the slip ring assembly in FIGS. 1-4.

As shown in FIG. 5, the slip ring assembly 500 may include a first slip ring 510, a second slip ring 520, and a transmission component 530. In some embodiments, the transmission component 530 may be operably connected to the first slip ring 510 and the second slip ring 520. The transmission component 530 may be configured to transmit at least one signal of the first slip ring 510 and at least one signal of the second slip ring 520.

As shown in FIG. 5, the transmission component 530 may include a static ring. The static ring may be located between the first slip ring 510 and the second slip ring 520. A radius of the static ring may be larger than the first slip ring 510, and a radius of the second slip ring 520 may be larger than the static ring. In some embodiments, the first slip ring 510, the second slip ring 520, and the static ring may be located in a same plane. More descriptions of the slip ring assembly 500 may be found elsewhere in the present disclosure, for example, the descriptions of the slip ring assembly in FIGS. 1-4.

FIG. 6A and FIG. 6B illustrate two views of an exemplary slip ring assembly according to some embodiments of the present disclosure. FIG. 6C illustrates a section view of the exemplary slip ring assembly along AA axis in FIG. 6A according to some embodiments of the present disclosure. The slip ring assembly 600 may be an example of the slip ring assembly in FIGS. 1-5. FIG. 6A is a section view of the slip ring assembly 600 in the xz plane defined by the x-axis and the z-axis illustrated in FIG. 1. FIG. 6B is a section view of the slip ring assembly 600 in the yz plane defined by the y-axis and the z-axis illustrated in FIG. 1.

As shown in FIGS. 6A-6C, the slip ring assembly 600 may include a first slip ring 610, a second slip ring 620, and a transmission component 630. In some embodiments, the transmission component 630 may be operably connected to both the first slip ring 610 and the second slip ring 620. The transmission component 630 may be configured to transmit at least one signal of the first slip ring 610 and at least one signal of the second slip ring 620.

In some embodiments, the transmission component 630 may include a static ring. The static ring may be located between the first slip ring 610 and the second slip ring 620. A radius of the static ring may be larger than the first slip ring 610, and a radius of the second slip ring 620 may be larger than the static ring.

In some embodiments, the first slip ring 610 may include channels 613. Each channel 613 is illustrated as a circle of the concentric circles of the first slip ring 610 in FIG. 6A. The second slip ring 620 may include channels 623. Each channel 623 is illustrated as a circle of the concentric circles of the second slip ring 620 in FIG. 6A. In some embodiments, the channels 613 may be used to implement first contact data transmission between the first slip ring 610 and the transmission component 630. The channels 623 may be used to implement second contact data transmission between the second slip ring 620 and the transmission component 630.

In some embodiments, a printed circuit board (PCB) 640 may be located on the first slip ring 610. A first carbon brush assembly (not shown in FIG. 6) may be located on the PCB 640. The first carbon brush assembly may be configured to facilitate first data transmission between the first slip ring 610 and the transmission component 630. In some embodiments, a PCB 650 may be located on the second slip ring 620. A second carbon brush assembly (not shown in FIG. 6) may be located on the PCB 650. The second carbon brush assembly may be configured to facilitate second data transmission between the second slip ring 620 and the transmission component 630. More descriptions of the slip ring assembly 500 may be found elsewhere in the present disclosure, for example, the descriptions of the slip ring assembly in FIGS. 1-5 and FIG. 7.

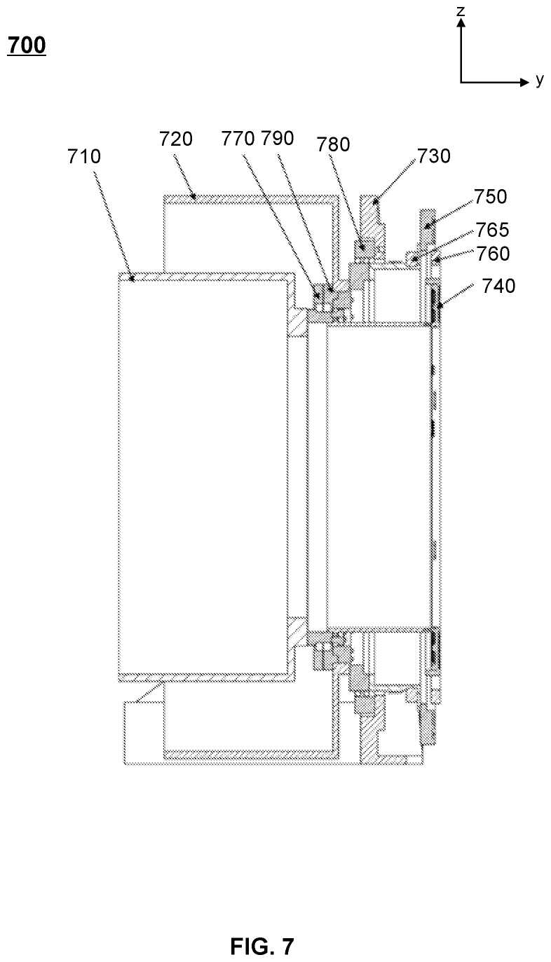

FIG. 7 is a section view illustrating an exemplary medical device according to some embodiments of the present disclosure. The medical device 700 may be an example of the medical device 110 in FIG. 1.

As shown in FIG. 7, the medical device 700 may include a first gantry 710, a second gantry 720, a fixed gantry 730, and a slip ring assembly. The first gantry 710 may be configured to accommodate at least a first portion of a first medical assembly (e.g., an imaging assembly, a treatment assembly) and at least a first portion of a second medial assembly (e.g., an imaging assembly, a treatment assembly). The second gantry 720 may be configured to accommodate at least a second portion of the first medical assembly and at least a second portion of the second medical assembly. The fixed gantry 730 may be configured to support the first gantry 710 and the second gantry 720. As shown in FIG. 7, at least a portion of the first gantry 710 may be housed in the second gantry 720.

As shown in FIG. 7, the medical device 700 may include a bearing 770 and a bearing 780. The first gantry 710 may be operably connected to the second gantry 720 through the bearing 770. The second gantry 720 may be operably connected to the fixed gantry 730 through the bearing 780.

In some embodiments, the slip ring assembly may include a first slip ring 740, a second slip ring 750, and a transmission component 760, and a transmission component 765. The first slip ring 740 and the second slip ring 750 may be arranged along an axial direction of the first slip ring 740 or an axial direction of the second slip ring 750. The transmission component 760 and the transmission component 765 may be arranged along an axial direction of the transmission component 760 or an axial direction of the transmission component 765. The transmission component 760 may be operably connected to the first slip ring 740. The transmission component 760 may be configured to transmit at least one signal of the first slip ring 740. The transmission component 765 may be operably connected to the second slip ring 750. The transmission component 765 may be configured to transmit at least one signal of the second slip ring 750.

In some embodiments, the first slip ring 740 may be operably connected to the first gantry 710. The second slip ring 750 may be operably connected to the second gantry 720. Each of the transmission component 760 and the transmission component 765 may be operably connected to the fixed gantry 730, respectively. Each of the transmission component 760 and the transmission component 765 may be located on the fixed gantry 730, respectively.

In some embodiments, the first slip ring 740 may be configured to supply power to and/or facilitate data transmission of at least one component (e.g., the first portion of the imaging assembly, the first portion of the treatment assembly) located on the first gantry 710. For example, data to be transmitted may include imaging data of a subject, treatment data of the subject, etc. In some embodiments, the second slip ring 750 may be configured to supply power to and/or facilitate data transmission of at least one component (e.g., the second portion of the imaging assembly, the second portion of the treatment assembly) located on the second gantry 720. For example, data to be transmitted may include imaging data of a subject, treatment data of the subject, etc.

In some embodiments, the first gantry 710 may be configured to rotate independently from the second gantry 720. In some embodiments, the first gantry 710 and the second gantry 720 may be configured to rotate synchronously. As shown in FIG. 7, the medical device 700 may include a lock mechanism 790. In some embodiments, the first gantry 710 and the second gantry 720 may be locked through the lock mechanism 790, and the first gantry and the second gantry may rotate synchronously. In some embodiments, the first gantry and the second gantry are unlocked through the lock mechanism 790, the first gantry 710 may rotate independently from the second gantry 720. More descriptions of the medical device 700 may be found elsewhere in the present disclosure, for example, the descriptions of the medical device 110 in FIG. 1.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A non-transitory computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A slip ring assembly comprising:
a first slip ring;
a second slip ring; and
a transmission component configured to facilitate at least one of
data transmission of the first slip ring,
data transmission of the second slip ring,
power transmission of the first slip ring, or
power transmission of the second slip ring.

2. The slip ring assembly of claim 1, wherein
the transmission component is configured to transmit at least one signal of the first slip ring and at least one signal of the second slip ring.

3. The slip ring assembly of claim 1, wherein the transmission component includes a static ring.

4. The slip ring assembly of claim 3, wherein at least two of the first slip ring, the second slip ring, or the static ring are located in a same plane and wind around each other.

5. (canceled)

6. The slip ring assembly of claim 3, wherein the static ring is located between the first slip ring and the second slip ring, a radius of the static ring is larger than a radius of the first slip ring; and a radius of the second slip ring is larger than the radius of the static ring.

7. (canceled)

8. The slip ring assembly of claim 2, wherein
the transmission component includes a first carbon brush assembly and a second carbon brush assembly;
the first carbon brush assembly is operably connected to the first slip ring and configured to facilitate at least one of first data transmission or first power transmission between the first slip ring and the transmission component; and
the second carbon brush assembly is operably connected to the second slip ring and configured to facilitate at least one of second data transmission or second power transmission between the second slip ring and the transmission component.

9. The slip ring assembly of claim 2, further comprising a carbon brush assembly, wherein the carbon brush assembly may be configured to facilitate
at least one of first data transmission or first power transmission between the first slip ring and the transmission component, and
at least one of second data transmission or second power transmission between the second slip ring and the transmission component.

10. (canceled)

11. The slip ring assembly of claim 2, wherein the transmission component includes at least one transmission module and at least one receiving module that are configured to facilitate
first contactless data transmission between the first slip ring and the transmission component, or
second contactless data transmission between the second slip ring and the transmission component.

12-13. (canceled)

14. The slip ring assembly of claim 11, wherein
one transmission module of the at least one transmission module is opposingly positioned with respect to one receiving module of the at least one receiving module that corresponds to the transmission module.

15-19. (canceled)

20. The slip ring assembly of claim 1, further comprising a second transmission component, wherein
the transmission component is configured to transmit at least one signal of the first slip ring; and
the second transmission component is configured to transmit at least one signal of the second slip ring.

21. The slip ring assembly of claim 20, wherein
the transmission component includes at least one carbon brush assembly; and
the at least one carbon brush assembly is operably connected to the first slip ring and the second slip ring; and
the at least one carbon brush assembly is configured to facilitate
at least one of first data transmission or first power transmission between the first slip ring and the transmission component, and
at least one of second data transmission or second power transmission between the second slip ring and the transmission component.

22. The slip ring assembly of claim 20, wherein
the transmission component includes a first static ring;
the second transmission component includes a second static ring that is different from the first static ring; and
at least two of the first slip ring, the second slip ring, the first static ring, or the second static ring are disposed coaxially.

23-26. (canceled)

27. A system comprising:
a first gantry configured to accommodate at least a portion of a first imaging assembly;
a second gantry configured to accommodate at least a portion of a second imaging assembly or at least a portion of a treatment assembly;
a fixed gantry configured to support the first gantry or the second gantry; and
a slip ring assembly configured to facilitate data transmission of the first imaging assembly, the second imaging assembly, or the treatment assembly, wherein
the slip ring assembly includes a first slip ring located on the first gantry;
the slip ring assembly includes a second slip ring located on the second gantry;
the slip ring assembly includes a transmission component located on
the fixed gantry; and
the transmission component is configured to faciliate
data transmission of the at least a portion of the first imaging assembly,
data transmission of the at least a portion of the second imaging assembly or the at least a portion of the treatment assembly,
power transmission of the at least a portion of the first imaging assembly, or
power transmission of the at least a portion of the second imaging assembly or the at least a portion of the treatment assembly.

28-30. (canceled)

31. The system of claim 27, wherein at least two of the first slip ring, the second slip ring, or the transmission component are located in a same plane and wind around each other.

32-33. (canceled)

34. The system of claim 27, further comprising a second transmission component, wherein
the transmission component is configured to transmit at least one signal of the at least a portion of the first imaging assembly; and
the second transmission component is configured to transmit at least one signal of the at least a portion of the first imaging assembly or the at least a portion of the treatment assembly.

35-37. (canceled)

38. The system of claim 27, wherein
at least a portion of the first gantry is housed in the second gantry;
the first gantry is rotatably connected to the second gantry; and
the second gantry is rotatably connected to the fixed gantry.

39. The system of claim 27, wherein
the first gantry is rotatable along a first axis;
the second gantry is rotatable along a second axis; and
the first axis intersects the second axis or the first axis is parallel to the second axis.

40-41. (canceled)

42. A method for operating a system, the system comprising a first medical assembly, a second medical assembly, and a slip ring assembly, wherein
the slip ring assembly is configured to facilitate data transmission of the first medical assembly, data transmission of the second medical assembly, power transmission of the first slip ring assembly, or power transmission of the second slip ring assembly, the method comprising:
obtaining data of a first portion of the first medical assembly or a first portion of the second medical assembly; and
transmitting, through the slip ring assembly, the data to a second portion of the first medical assembly, a second portion of the second medical assembly, or a control component of the system.

43. The method of claim 42, wherein the data includes imaging data of a subject or treatment data of the subject acquired by the first medical assembly or the second medical assembly.

44. The method of claim 42, wherein the transmitting, through the slip ring assembly, the data to a second portion of the first medical assembly, a second portion of the second medical assembly, or a control component of the system includes:
transmitting the data through a contact data transmission mode; or
transmitting the data through a contactless data transmission mode.

45. (canceled)
